(19) United States
(12) Patent Application Publication
(10) Pub. No.: US 2015/0147306 A1
(43) Pub. Date: May 28, 2015

(54) COMPOSITION FOR TREATING A CIRCADIAN RHYTHM DISORDER

(71) Applicant: Jürgen Ruhlmann, (US)

(72) Inventors: Jürgen Ruhlmann, Bonn (DE); Benjamin Jürgen Ruhlmann, Bonn (DE)

(73) Assignee: Jürgen Ruhlmann, Bonn (DE)

(21) Appl. No.: 14/401,713

(22) PCT Filed: May 22, 2013
(86) PCT No.: PCT/EP2013/060541

§ 371 Date: Nov. 17, 2014

(30) Foreign Application Priority Data
May 23, 2012 (DE) 10 2012 104 451.1

(51) Int. Cl. A61K 31/7084; A61K 31/714; A61K 31/455; A61K 31/525; A23L 1/30; A61K 31/194; A61K 31/405; A61K 31/197; A61K 31/122; A61K 31/7004; A61K 31/375

(52) U.S. Cl. CPC A61K 31/7084; A61K 31/7004; A61K 31/714; A61K 31/455; A61K 31/525; A61K 31/375; A61K 31/194; A61K 31/405; A61K 31/197; A61K 31/122; A23L 1/30; A23V 2002/00
USPC 424/94.1; 514/47

(57) ABSTRACT

The invention relates to a composition comprising NADH and D-galactose for treating a circadian rhythm disorder, in particular further comprising vitamins and/or coenzymes and/or minerals.

The present invention relates to a composition comprising NADH and D-galactose for treating a circadian rhythm disorder and relates in particular to the use of a specific combination of vitamins, coenzymes, minerals and galactose to facilitate synchronisation of a circadian rhythm, in particular jet-lag.

Circadian rhythm disorders are caused primarily by time differences brought about by long-haul flights, shift work, work under extreme conditions including artificial sleep-wake rhythms.

It is known to administer melatonin as a medicament to enable more rapid adaptation to time differences, e.g. after long-haul flights. Melatonin is a tryptophan derivative and is produced by the so-called pineal gland. In the pinealocytes, which are the cells of this gland, tryptophan from the blood is converted by several biosynthesis steps to produce melatonin. In the USA, melatonin is authorised as a foodstuff but not as a drug. In the Federal Republic of Germany, melatonin preparations are not available on the free market, and are only available in the form of the drug “Circadin”. The combination of the aforementioned substances stimulates inter alia the formation of melatonin. As far as is known to date, melatonin has the above-described effect by reason of the following mechanistic attempted explanations: the melatonin circulating in the bloodstream acts mainly at central binding sites in specific areas of the hypothalamus and the adenohypophysis. As far as is known, the melatonin concentrations in human serum demonstrate a circadian pattern with high concentrations at night and low concentrations during the day. This pattern is produced by the symmetric innervation of the pineal organ. Mammals perceive the daily change from light to dark through the retina. This information is then relayed by neurons via the retinohypothalamic tract to the suprachiasmatic nucleus (SCH), the so-called circadian clock. After further switching points, postganglionic sympathetic fibres which originate in the superior cervical ganglion (SCG) reach the pineal organ from this location. During the dark phase, these fibres are stimulated by the activity of the SCH which causes a release of the neurotransmitter noradrenalin at the nerve endings located in the pineal organ. Noradrenalin interacts with predominantly β-adrenergic receptors on the membrane of pinealocytes and stimulates the activity of the N-acetyl transferase via the cAMP second messenger system. According to current knowledge, this reaction constitutes the rate-determining step of the melatonin synthesis.

The above-described melatonin system is subjected to light as time tissue which synchronises the circadian rhythm. Exposure to light causes the distribution/discharge of noradrenalin to cease. This results in inhibition of melatonin synthesis followed by a decrease in the concentration of melatonin in the blood. In humans, melatonin in the serum has a half-life of about 35 to 50 minutes. Melatonin thus acts as an endocrine signal for the length of the night because the period during which melatonin is released during the night is proportional to the length of the dark phase. The organism is thus able to determine the respective time of day and year it is currently experiencing with the aid of the melatonin profile. In humans, who demonstrate less pronounced seasonal adaptations than animals, melatonin is primarily involved in regulating the sleep-wake cycle, for which reason melatonin is used successfully to facilitate adaptation after long-haul flights, i.e. in the case of so-called jet-lag (ARENDT J., ALDHOUS M., MARKS M. (1987): Some effects of jet-lag and their treatment by melatonin; Ergonomics 30: 1393-1397 (1987); ARENDT J., ALDHOUS M., MARKS V. (1986): Alleviation of jet-lag by melatonin: preliminary results of controlled double-blind trial, BMJ 292: 1170).

It is known from US 2003/0021772 A1 to use NADH exclusively to alleviate disorders based on sleep deprivation or jet-lag.

The object of the present invention is to provide a possible alternative to melatonin or NADH exclusively as a means for adapting to an external zeitgeber.

This object is achieved by the composition described in claim 1.

In accordance with the invention, it has been recognised that a composition comprising NADH and D-galactose is suitable for treatment of a circadian rhythm disorder and is suitable for facilitating synchronisation of the circadian rhythm by means of an external zeitgeber, such as the change from light to dark, day-night rhythm changes, time differences which occur in the case of long-haul flights and so-called jet-lag.

However, the use of the aforementioned composition is also suitable for use e.g. in the case of forced changes to the rhythm of activity experienced by shift workers.

NADH is a coenzyme and the energy-rich, reduced form of NAD+. In the oxidative metabolism it serves as a energy-supplying coenzyme of the respiratory chain, wherein ATP is generated. During oxidation of the NADH, it gives off electrons and thus transmits them to oxygen during the intracellular knallgas reaction, thus producing NAD+ and water. NAD+ is inter alia also a coenzyme of e.g. the alcohol hydrogenase which oxidises alcohol. NAD+ is produced in the body on the one hand from niacin (vitamin B3, nicotinic acid) or nicotinamide, and is produced on the other hand from the decomposition products of the amino acid tryptophan.

These two starting substances are essential for achieving deficiency symptoms of NAD+, which, however, is rare owing the redundancy of both metabolic pathways.

By reason of the nowadays rather reduced supply of niacin and tryptophan, low-normal NAD+ levels occur more frequently.

NADH is one of the strongest antioxidants, the effect is intended on the one hand to impede or slow down the oxidation processes in the body, i.e. degradation processes due to the reaction with oxygen, and is intended on the other hand to prevent aggressive or harmful substances from the environment from being absorbed by the cell. NADH plays a crucial role above all in energy generation, it is the most important electron transporter within the energy-producing processes. Without the effect of NADH, a human cannot mobilise his energy in an optimum manner. He feels weak, tired, even exhausted. By reason of our current lifestyle/diet, the supply of NADH is no longer adequately ensured, e.g. most of the NADH effect is already lost through meat and vegetables being cooked.

The more NADH is provided to the cell, the more energy the cell can produce.

NADH stimulates or accelerates the production of the body's own “happy hormones” dopamine and noradrenalin. This has a significant effect upon the power of concentration and individual performance as well as upon general prevailing mood, motivation and individual energy potential, the improvement of each performance is improved. NADH reduces jet-lag, can compensate for a lack of sleep and can also increase libido. Since NADH very effectively combats free radicals, body cells are also protected in a sustainable manner (literature Prof. Dr. J. Birkmayer: NADH (Koenzym 1), biologische Funktion and therapeutische Anwendungen [NADH (coenzyme 1), biological function and therapeutic applications] DVD-Wissen.com).

Galactose is a naturally occurring simple sugar. In contrast to glucose, galactose can penetrate independently of insulin via a concentration gradient into the cell, above all the brain cell where galactose is rapidly and completely converted into glucose thus improving brain function.

In particular, it has been found that the composition is particularly effective if it further comprises a least one or a plurality of substances from the group consisting of L-tryptophan, nicotinic acid or nicotinamide (vitamin B3), vitamin B2, vitamin B12, magnesium, L-carnitine, coenzyme Q10, vitamin C or precursors of the aforementioned substances.

Precursors are understood in particular to mean “preliminary stages” of the said substances which are converted by the body into the said substances.

Tryptophan is an aromatic amino acid and constituent of proteins and peptides. It cannot be produced in the human organism which is dependent on it being supplied in food. L-tryptophan is the precursor of the serotonin. Stress—and the cortisol level increased to a limited extent thereby—results in activation of the tryptophan-degrading enzyme tryptophan pyrrolase. It is not very easy to reach an overdose of L-tryptophan, as L-tryptophan itself is the main activator of its degrading enzyme tryptophan pyrrolase.

Niacin, which is also called nicotinic acid, is a B-complex vitamin. It is found in all living cells and forms an important building block of various coenzymes, in this form it is of significant importance for the metabolism of proteins, fats and carbohydrates. Nicotinic acid has an antioxidative effect and is inter alia also important for the regeneration of skin muscles, nerves and DNA. The average daily requirement for women is 13 to 15 mg, and for men it is 15 to 20 mg.

Coenzyme Q10, or also ubiquinone 10, is absorbed in part from food but is also produced in the body itself. It is involved as a coenzyme in the oxidative phosphorylation, over 95% of all of the body's energy (ATP) is produced with the “controlled knallgas reaction”. The electrons for reducing the ubiquinone originate from the oxidation of the NADH.

Vitamin B complex: this group of vitamins includes eight vitamins which all serve as preliminary stages for coenzymes. They are B1 thiamine, B2 riboflavin, B6 pyridoxine, B12 cobalamin, B7 biotin, B9 folic acid, B3 nicotinic acid and B5 pantothenic acid.

Vitamin C is a radical interceptor and has an antioxidative effect, it constitutes an important coenzyme for an enzyme during the biosynthesis of the protein collagen. Moreover, it converts proline residues into hydroxyproline which is absolutely essential for stable collagen formation. Vitamin C is an important cofactor in the hydroxylation of steroids and it also plays an important role in the formation of amino acids, such as L-thyrosine. In addition, it is necessary for the conversion of dopamine to noradrenalin, in the metabolism of cholesterol and in the biosynthesis of carnitine. With niacin and vitamin B6, vitamin C controls the production of L-carnitine which is required for burning fat in musculature. It also promotes iron resorption in the small intestine.

Magnesium is essential for all organisms. It is involved in about 300 enzyme reactions as an enzyme constituent or coenzyme. Magnesium ions functions as a second messenger in the immune system. Magnesium deficiency in humans also triggers inter alia headaches, deficiency in concentration, tiredness, a general feeling of weakness, cardiac rhythm disorders and muscle cramps. A slight deficiency can occur inter alia in competitive sports.

Up to now, it is known how the individual aforementioned substances act in their own right. However, it is still not yet known that the combination of said substances rapidly eliminates the most common complaints of jet-lag, such as tiredness, feeling of dizziness, mood fluctuations, loss of appetite, reduced performance in terms of physical, manual and cognitive requirements and sleep disorders.

Vitamin B2, vitamin B12, vitamin B3, magnesium, coenzyme Q10, tryptophan are necessary substances which activate the mitochondrial metabolism. For example, in the respiratory chain there are 3 to 5 reactions involving Q10. Without magnesium, ATP cannot be formed. Carnitine is required at the mitochondrial membrane for stabilisation purposes and fats can only be introduced into the mitochondrion with the aid of carnitine, thus achieving improved energy metabolism.

Galactose is introduced into the metabolism as an immediately effective carbohydrate, independently of insulin.

Vitamin C also acts on all levels as an antioxidant.

Since the brain has few protective factors, it is protected against oxidative processes by Q10, vitamin C.

Preferably, the external change from light to dark includes: day-night rhythm, time differences, in particular after long-haul flights, preferably jet-lag, social zeitgebers, in particular forced changes to the activity rhythm during shift work.

The composition is very suitable for producing a food supplement, in a solid, liquid or powder form.

The corresponding food supplement containing the combination in accordance with the invention is suitable to be taken for treating a circadian rhythm disorder.

In a particularly preferred embodiment, the individual components of the composition are present in the following mol ratios:

| Component | Ratio |
|---|---|
| NADH | 91-121 |
| Vitamin B2 | 537-716 |
| Vitamin B3 | 125-166 |
| Vitamin B12 | 1 |
| Vitamin C | 1912-3059 |
| Galactose | 22450-29933 |
| Magnesium citrate | 220-439 |
| Tryptophan | 990-2641 |
| Carnitine | 2510-5020 |
| Q10 activated | 78-125 |

A particularly preferred embodiment of the composition in accordance with the invention comprises the following quantity ranges of the individual components:

| | | |
|---|---|---|
| NADH | 30-90 | mg |
| Vitamin B2 | 100-300 | mg |
| Vitamin B3 | 50-150 | mg (inositol nicotinate - retarded action) |
| Vitamin B12 | 500-2000 | μg (as hydroxycobalamin) |
| Vitamin C | 200-500 | mg |
| Galactose | 2-6 | g |
| Magnesium citrate | 100-200 | mg |
| Tryptophan | 200-500 | mg |
| Carnitine | 300-600 | mg |
| Q10 activated | 40-100 | mg |

For the purpose of reducing the symptoms of jet-lag, the administration of the following substances comprising the following concentration has proven to be a particularly preferred dosage:

| | | |
|---|---|---|
| NADH | 30 | mg |
| Vitamin B2 | 200 | mg |
| Vitamin B3 | 100 | (as inositol nicotinate - retarded action) |
| Vitamin B12 | 1000 | μg (as hydroxycobalamin) |
| Vitamin C | 250 | mg |
| Galactose | 4 | g |
| Magnesium citrate | 150 | mg |
| Tryptophan | 250 | mg |
| Carnitine | 500 | mg |
| Q10 activated | 40 | mg |

In a quite particularly preferred manner, these components and quantities are dissolved in at least 30 ml liquid, in particular water, quite particularly preferably 60 ml.

The efficacy of the aforementioned combination was demonstrated on test volunteers by the use in accordance with the invention for facilitating synchronisation of the circadian rhythm by an external zeitgeber.

The use in accordance with the invention was tested on human test volunteers on long-haul flights from the Federal Republic of German to the USA and China.

The dosage was administered from a biochemical viewpoint and depending on the recommendation of the commercially available preparations.

It was subjectively reported that all of the test persons became accustomed to the time difference from −9 hours (Los Angeles, Las Vegas) to +7 hours (Hong Kong) within one to two days, which was expressed on the one hand by sleeping rhythms adapted to the new time and on the other hand by a significant increase in performance in terms of physical, (manual) and cognitive requirements, rapid decrease in tiredness and loss of appetite already about half an hour to one hour after taking the inventive preparation combination.

All of the test persons were subjected to a time difference as a result of a long-haul flight from Frankfurt to Los Angeles (−9 hours) and ten further test persons who were subjected to a time difference of −7 hours (long-haul flight from Frankfurt to Hong Kong) took the inventive combination and dosage of the aforementioned preparations in each case upon arrival at the destination and the next morning after breakfast.

All of the test persons spent at least five days at their destination. After returning, they again took the inventive combination of the preparations in each case directly upon arrival and also after breakfast on the following day.

They reported consistently and independently that they adapted to the new time without any trouble within one to two days and that they experienced a significant improvement in performance in terms of physical, manual and communicative requirements, a rapid decrease in tiredness and appetite returning to normal already half an hour to one hour after taking the inventive preparation combination both upon arrival and return.

In order to verify the efficacy of the combination of NADH and galactose, in a similar manner to the above procedure, the use in accordance with the invention for facilitating synchronisation of a circadian rhythm by means of an external zeitgeber was demonstrated on test volunteers.

To this end, the use of a combination of 40 mg NADH and 4g galactose (also with flavouring additives to improve taste) was tested in turn on 23 human test volunteers taking long-haul flights from the Federal Republic of Germany to the USA and Asia.

All of the test persons complained about feelings of sickness brought about by jet-lag. To this end, the sleep disorders, increased tiredness, feelings of dizziness, fluctuations in mood, los of appetite, reduced mental and physical performance were evaluated subjected using a scale of 1 (very low to non-existent) to 6 (very high). The rating of the disorders fluctuated between 3 and 6 on the scale. Only the loss of appetite fluctuated between 1 and 3 on the scale.

All of the test persons each took the stated combination of NADH and galactose once upon arrival at their destination.

They reported consistently and independently that they experienced an improvement in feelings of sickness by 1 to 2 points on the scale.

1-6. (canceled)

7. A method for treating a circadian rhythm disorder, comprising:
administering to a subject in need thereof an effective amount of a composition comprising NADH and D-galactose.

8. The method of claim 7, wherein the composition further comprises a vitamin, coenzyme or mineral, or a combination thereof.

9. The method of claim 7, wherein the composition further comprises at least one selected from the group consisting of L-tryptophan, nicotinic acid or nicotinamide, vitamin B12, vitamin B2, L-carnitine, magnesium, coenzyme Q10, vitamin C, and a precursor of one of the aforementioned.

10. The method of claim 7, wherein the composition comprises NADH, vitamin B2, vitamin B3, vitamin B12, vitamin C, galactose, magnesium citrate, tryptophan, carnitine, and coenzyme Q10.

11. The method of claim 7, wherein the composition comprises: 30-90 mg NADH, 100-300 mg vitamin B2, 50-150 mg vitamin B3, 500-2000 μg vitamin B12, 200-500 mg vitamin C, 2-6 g galactose, 100-200 mg magnesium citrate, 200-500 mg tryptophan, 300-600 mg carnitine, and 40-100 mg coenzyme Q10.

12. The method of claim 11, wherein the composition comprises: 30 mg NADH, 200 mg vitamin B2, 100 mg vitamin B3, 1000 μg vitamin B12, 250 mg vitamin C, 4 g galactose, 150 mg magnesium citrate, 500 mg tryptophan, 500 mg carnitine, and 100 mg coenzyme Q10.

13. The method of claim 7, wherein the composition is administered as a food supplement.

14. The method of claim 7, wherein the composition is in a liquid form.

15. The method of claim 14, wherein the composition comprises at least 30 ml water.

16. The method of claim 7, wherein the circadian rhythm disorder is caused by jet-lag.

17. The method of claim 7, wherein the circadian rhythm disorder is caused by a social zeitgeber.
